(19) United States
(12) Patent Application Publication
(10) Pub. No.: US 2022/0241407 A1
(43) Pub. Date: Aug. 4, 2022

(54) MINI-COMBINED ADJUVANTS CARRIER-FREE NANOPARTICLES AND REPARATION METHOD AND APPLICATION THEREOF

(71) Applicant: INSTITUTE OF BIOMEDICAL ENGINEERING, CHINESE ACADEMY OF MEDICAL SCIENCES, TIANJIN (CN)

(72) Inventors: LANXIA LIU, TIANJIN (CN); DAN LIU, TIANJIN (CN); JIALE LIU, TIANJIN (CN); XIGANG LENG, TIANJIN (CN)

(73) Assignee: INSTITUTE OF BIOMEDICAL ENGINEERING, CHINESE ACADEMY OF MEDICAL SCIENCES, TIANJIN (CN)

(21) Appl. No.: 17/256,095

(22) PCT Filed: Jun. 17, 2020
(86) PCT No.: PCT/CN2020/096576

§ 371 Date: Dec. 24, 2020

(30) Foreign Application Priority Data
Oct. 23, 2019 (CN) 201911012900.7

(51) Int. Cl. A61K 39/39; A61P 31/06; A61P 35/00

(52) U.S. Cl. CPC A61K 39/39; A61K 2039/55555; A61P 35/00; A61P 31/06

(57) ABSTRACT

The present disclosure provides combined adjuvants nanoparticles prepared by self-assembly with amphiphilic monomer molecules as the building blocks, wherein the amphiphilic monomer molecules are generated from the reaction between hydrophobic adjuvants molecules and hydrophilic adjuvants molecules.

This application claims priority to Chinese Patent Application No. 201911012900.7, entitled “Mini-combined adjuvants nanoparticles and preparation method and application thereof”, filed to China National Intellectual Property Administration on Oct. 23, 2019, the entire contents of which are incorporated herein by reference.

### TECHNICAL FIELD

The present disclosure belongs to the field of biotechnology, and specifically pertains to combined adjuvants nanoparticles prepared by self-assembly and the preparation method and application thereof.

### BACKGROUND

Adjuvants are important component of vaccines and usually used to inject with antigens to enhance the immune response or polarize the type of the immune response. They have no immunogenicity in themselves, but can nonspecifically stimulate the immune system. At present, adjuvants commonly used in the field of vaccines include aluminum salts, oil emulsion, propolis, polysaccharides, microbial Freund's Adjuvant (FA), γ-interferon (IFN-γ), Interleukins (ILs), immunostimulating complexes (ISCOMs), glycosides, compound traditional Chinese medicine adjuvants and so on. Novel adjuvants licensed or in development include nucleic acid, CpG, complements, nanometer, liposomes (LIP) or the combined adjuvants of two or more adjuvants. Currently, in order to enhance the efficacy of vaccines, a nano-delivery system is often needed to deliver antigens and/or adjuvants to induce stronger protective immune responses, and the delivery system is generally prepared with biomaterials; however, Nanomaterials-based nanovaccine delivery system limit their clinical application in immunotherapy due to potential biosecurity concerns, inefficient loading and complicated preparation process. So, it is desirable to develop a highly efficient nanocarriers using adjuvants and/or antigens themselves without extra materials involved which have not therapeutic function, which could achieve the optimal therapeutic effects and minimize the potential side reactions.

### SUMMARY

With respect to defects in the prior art, the present disclosure provides combined adjuvants carrier-free nanoparticles.

In one aspect, the present disclosure provides combined adjuvants carrier-free nanoparticles, the combined adjuvants nanoparticles are prepared with amphiphilic monomer molecules by self-assembly, the amphiphilic monomer molecules are generated from the reaction between hydrophobic adjuvants molecules and hydrophilic adjuvants molecules.

In some embodiments, the combined adjuvants carrier-free nanoparticles have diameters of 100-200 nm.

In some embodiments, the hydrophobic adjuvants molecules are monophosphatidyl lipid A or analogues thereof.

In some embodiments, the hydrophilic adjuvants molecules are oligonucleotides or oligodeoxynucleotides. Preferably, the hydrophilic adjuvants molecules are CPG−ODN.

In another aspect, the present disclosure provides a preparation method for the combined adjuvants nanoparticles, comprising the following steps:

S1: The monophosphatidyl lipid A or analogues thereof are modified with azide groups, and then subjected to dialysis and lyophilization;

S2: The resulting substances from S1 are mixed with oligodeoxynucleotides by stirring at room temperature for 12-18 h, and then subjected to dialysis and lyophilization to get the combined adjuvants nanoparticles.

In some embodiments, diphenylphosphoryl azide and 1,8-diazabicyclo[5.4.0]undec-7-ene are added for modification with azide groups, they are reacted with stirring at 20° C. for 24˜48 h and then subjected to dialysis and lyophilization; the ratio of the mass of monophosphatidyl lipid A, the volume of diphenylphosphoryl azide, and the volume of 1,8-diazabicyclo[5.4.0]undec-7-ene is (2.0˜4.0) mg:(3.0˜6.0) μl:(2.0˜4.0) μl.

In some embodiments, in step S2, the ratio of the mass of the resulting substances from S1 to the volume of oligodeoxynucleotides is (1.0˜2.0) mg:(100˜200) μl.

Specifically, dialysis is conducted with dialysis bags, and substances with higher molecular weight are collected during dialysis.

In some embodiments, the substances loaded are selected from drugs and antigens. They are preferably chicken ovalbumin. Of course, they also may be other antigens drugs.

In a further aspect, the present disclosure provides an application of the combined adjuvants carrier-free nanoparticles in the preparation of a complex loaded with drugs and antigens.

In a further aspect, the present disclosure provides an immunogenic composition, which contains effective amounts of antigens and the above combined adjuvants nanoparticles.

In some embodiments, the resulting substances from S2 are mixed with the substances to be loaded, and they are reacted with stirring at room temperature for 8-10 h to get immunogenic composition nanoparticles. The substances to be loaded may be chicken ovalbumin or other antigens or drugs. When the substances to be loaded are chicken ovalbumin, the mass ratio of the resulting substances from S2 to the substances to be loaded is 1:1-2.

The present disclosure further provides an application of the above composition in the preparation of vaccines for treating or preventing tumors or tuberculosis.

The present disclosure has the following beneficial effects:

The combined adjuvants carrier-free nanoparticles of the present disclosure are prepared by self-assembly with amphiphilic monomer molecules formed with hydrophilic adjuvants molecules and hydrophobic adjuvants molecules as building blocks. The stimulation effect of the combined adjuvants nanoparticles is stronger than that of hydrophilic adjuvants and hydrophobic adjuvants applied in combination in their free states. Moreover, the combined adjuvants nanoparticles can also be used as nanocarriers to deliver antigens to antigen-presenting cells, promote the uptake of antigens by antigen-presenting cells, co-deliver antigens and adjuvants, and produce synergistic immune responses, which can greatly enhance the immunotherapy of tumors and infectious diseases.

### BRIEF DESCRIPTION OF THE DRAWINGS

FIG. 1 shows the particle sizes of the combined adjuvants carrier-free nanoparticles detected by dynamic light scattering;

FIG. 2 is the typical image of the combined adjuvants carrier-free nanoparticles acquired with transmission electron microscope of;

FIG. 3 shows the cell viability of DC treated with various nanoparticles;

FIG. 4 shows the internalization of nanoparticles by DC cells;

FIG. 5 shows the maturation profile of DC cells induced by the nanoparticles;

FIG. 6 shows the activation profile of DC cells after incubation with nanoparticles.

### DETAILED DESCRIPTION

The embodiments of the technical solution of the present disclosure will be further described in detail accompany with the attached drawings. The following embodiments are only used to illustrate the technical solution of the present disclosure more clearly, therefore they are only exemplary and not intended to limit the protection scope of the present disclosure.

It should be noted that, unless otherwise noted, the technical terms or scientific terms used in this application shall have general meanings understood by the technical personnel in the field to which the present disclosure belongs.

The experimental methods in the following embodiments are conventional methods unless otherwise specified. The test substances used in the following embodiments, unless otherwise specified, were purchased from conventional biochemical reagent companies. The quantitative tests in the following embodiments were set to be repeated for three times, and the data were the mean of the three replications or the mean±standard deviation.

The present disclosure provides combined adjuvants carrier-free nanoparticles, which are prepared by self-assembly with amphiphilic monomer molecules as self-assembly building blocks, wherein the amphiphilic monomer molecules are generated from the reaction between hydrophobic adjuvants molecules and hydrophilic adjuvants molecules.

As one embodiment of the present disclosure, the hydrophilic adjuvants molecules are oligodeoxynucleotides CPG−ODN.

As one embodiment of the present disclosure, the hydrophobic adjuvants molecules are monophosphatidyl lipid A (MPLA) or analogues thereof.

As one embodiment of the present disclosure, the hydrophilic adjuvants molecules are oligodeoxyribonucleotides CPG−ODN. Preferably, they are type C 2395, the sequence of CPG−ODN (SEQ ID NO.1) is: 5′-TCGTCGTTTTCGGCGCGCGCCG-3′, and purchased from Sangon Biotech (Shanghai) Co., Ltd.

As one embodiment of the present disclosure, the hydrophobic adjuvants molecules are reacted with diphenylphosphoryl azide (DPPA) and 1,8-diazabicyclo[5.4.0]undec-7-ene (DBU) to modify the hydrophobic adjuvants molecules with azide groups.

When the hydrophobic adjuvants molecules are monophosphatidyl lipid A, the ratio of the mass of monophosphatidyl lipid A (MPLA) to the volumes of DPPA and DBU is: (2.0˜4.0) mg:(3.0˜6.0) μl:(2.0˜4.0) μl. The ratio of the mass of the substance generated from the azidation of monophosphatidyl lipid A to the volume of CPG−ODN is (1.0˜2.0) mg:(100˜200) μl.

### Embodiment 1

As shown in the formula below, it shows a synthetic route of monophosphatidyl lipid A (MPLA) and CPG−ODN (its sequence is SEQ ID NO.1) according to one embodiment of the

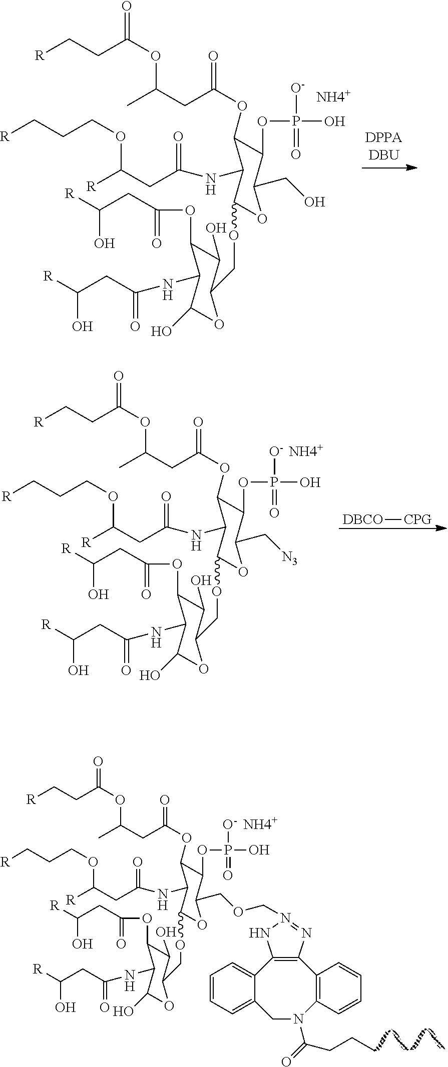

present disclosure:

Wherein,

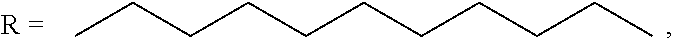

The combined adjuvants nanoparticles were prepared by the specific steps including:

S1: 4.0 mg monophosphatidyl lipid A (MPLA), 6.0 μl diphenylphosphoryl azide (DPPA) and 4.0 μl 1,8-diazabicyclo[5.4.0]undec-7-ene (DBU) were mixed and reacted with stirring at 20° C. for 48 h. The resulting products were dialyzed with dialysis bags, substances with molecular weight greater than 1000 kD were collected and lyophilized;

S2: 2.0 mg lyophilized substances were mixed with 200 μl functional oligodeoxynucleotide CPG−ODN and stirred at room temperature overnight, then dialyzed with dialysis bags, substances with molecular weight greater than 2000 kD were collected and lyophilized to get MPLA−CPG combined adjuvants nanoparticles.

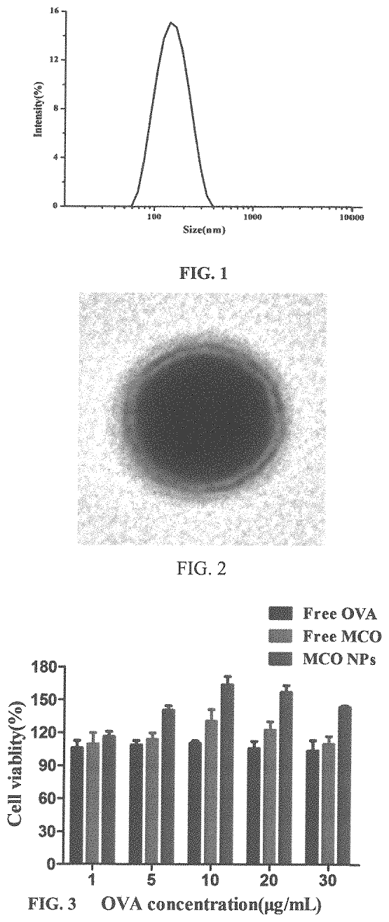

FIG. 1 shows the detection results of dynamic light scattering particle sizes of the combined adjuvants nanoparticles prepared in embodiment 1; FIG. 2 is the transmission electron micrograph of the combined adjuvants nanoparticles.

The resulting combined adjuvants nanoparticles have diameters of 136.9-138.6 nm. The dispersibility indexes of the combined adjuvants nanoparticles are 0.11-0.16.

### Embodiment 2

1.0 mg MPLA−CPG nanoparticles and 1.0 mg chicken ovalbumin (OVA) are reacted with stirring at room temperature for 10 h, to get MPLA−CPG−OVA nanoparticles.

### Embodiment 3

### 1. Cell Viability

Bone marrow derived dendritic cells (BMDC) were generated from 6˜8 week old C57BL/6 mice and induced with IL-4 and GM-CSF in an incubator at 37° C. and 5% CO₂. On day 7, the cells were collected and seeded in a 96-well plate, and placed in an incubator overnight. Each well was added MPLA−CPG nanoparticles loaded with OVA (chicken ovalbumin) at concentrations of 0, 1, 5, 10, 20, 30 μg/ml, respectively, and co-cultured for 24 hours. Then each well was added with 10 μl CCK-8 detection liquid, and continued cultivation in the incubator for 1˜4 h. A multimode microplate reader (ThermoVarioskan Flash3001) was used to determine the absorbance at 450 nm. Free OVA and a mixture of OVA+MPLA+CPG at the same concentration of OVA were used as the control. The cell viabilities of DC cells treated with various nanoparticles were shown in FIG. 3, wherein Free MPLA+CPG+OVA was indicated with Free MCO, and MPLA−CPG−OVA NPs was indicated with MCO NPs.

### 2. Cell Uptake

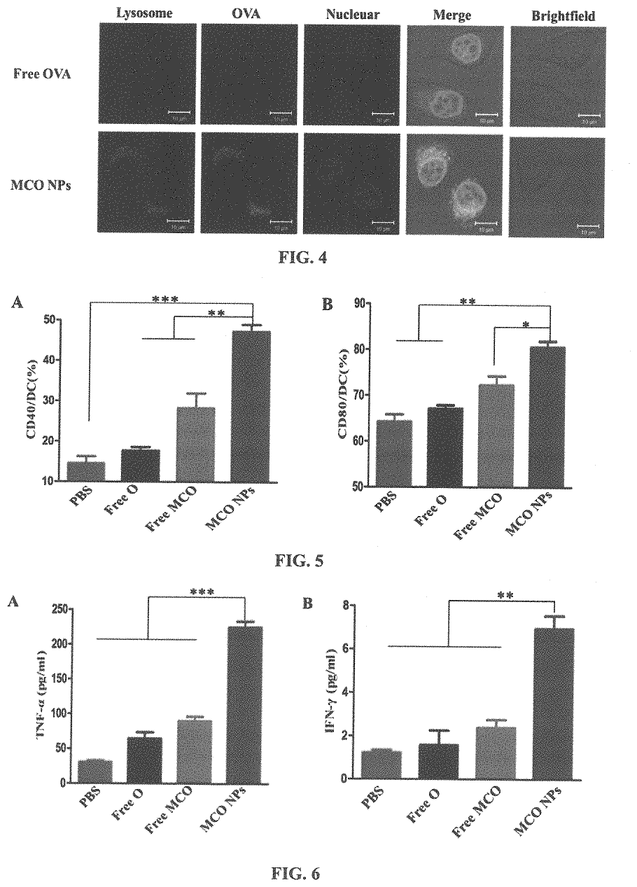

To Observe the uptake of nanoparticles by BMDCs, OVA was labelled with FITC, and the cells were plated in a confocal dish. BMDCs were co-incubated with MPLA−CPG nanoparticles loaded with FITC−OVA for 6 h (the concentration of OVA was at 10 μg/ml), washed with PBS, and fixed with a fixative solution. The lysosome was stained with Lyso-Tracker Red, and the cell nucleus was stained with DAPI. A laser scanning confocal microscope (Leica, TCS SP5) was used to observe the distribution of MPLA−CPG nanoparticles loaded with FITC−OVA in BMDCs, and the whole operation was performed in dark. Free FITC−OVA at the same concentration was used as the control. The confocal image of the uptake of nanoparticles by DC cells was shown in FIG. 4, wherein MPLA−CPG−OVA NPs was indicated with MCO NPs. The results showed that the uptake amount of MPLA−CPG−OVA nanoparticles by BMDCs was significantly higher than Free OVA.

### 3. Effects of Nanoparticles on the Maturation of BMDCs

The maturation of BMDCs induced by nanoparticles was determined by flow cytometry. BMDCs were co-incubated with MPLA−CPG nanoparticles loaded with OVA for 8 hours (the dosages were calculated based on the concentration of OVA at 10 μg/ml). Cells were collected, labelled with CD11C, CD40 and CD80 flow antibodies, and detected with a flow cytometer. PBS, the same concentration of free OVA and the mixture of OVA+MPLA+CPG were used as the control. The maturation results of DC cells induced by nanoparticles were shown in FIG. 5, wherein free OVA was indicated with Free O, Free MPLA+CPG+OVA was indicated with Free MCO, and MPLA−CPG−OVA NPs was indicated with MCO NPs.

The effect of nanoparticles on the cytokine secretion of BMDCs was determined by ELISA: BMDCs were collected and seeded in a 96 -well plate. BMDCs were co-incubated with MPLA−CPG nanoparticles loaded with OVA for 8 hours (the dosages were calculated based on the concentration of OVA at 10 μg/ml), and then the supernatant medium was discarded after centrifugation and replaced with a fresh medium and continued cultivation for 24 h. The contents of cytokines IFN-γ (Interferon-γ) and TNF-α (Tumor Necrosis Factor-α) in the supernatant of BMDCs were determined following the instruction of ELISA kit. The absorbance OD values at 450 nm were determined with a micro-plate reader. A standard curve was drawn according to the absorbance and concentrations of the standard to calculate the concentrations of samples. PBS, free OVA and a mixture of OVA+MPLA+CPG at the same concentration were used as the control. The cytokine secretion of DC cells after incubation with nanoparticles was shown in 6, wherein free OVA was indicated with Free O, Free MPLA+CPG+OVA was indicated with Free MCO, and MPLA−CPG−OVA NPs was indicated with MCO NPs. The results showed that MPLA−CPG−OVA nanoparticles can significantly promote the cytokine secretion of BMDCs, thus having a role of enhancing the activation of antigen-presenting cells.

It can be seen from the results of FIG. 3 to FIG. 6 that, the combined use of hydrophilic adjuvants molecules and hydrophobic adjuvants molecules in free states may also have synergistic stimulation effect, but the combined adjuvants nanoparticles prepared from the ligation of the two could produce greatly enhanced synergistic effect than that of the combination in their free states.

Finally, it should be noted that: the above embodiments are only intended to illustrate the technical solution of the present disclosure, rather than limitation. Although the present disclosure has been explained in detail with reference to the above embodiments, it should be understood to those of ordinary skills in the art that the technical solution documented in the above embodiments still may be modified, or part or all of the technical features thereof can be replaced equivalently. However, all these modifications or replacements which make the spirit of corresponding technical solution not deviating from the scope of technical scope of each embodiment in the present disclosure shall be covered within the scope of claims and description of the present disclosure.

1. Combined adjuvants carrier-free nanoparticles, wherein, the combined adjuvants nanoparticles are prepared with amphiphilic monomer molecules by self-assembly, the amphiphilic monomer molecules are generated from the reaction between hydrophobic adjuvants molecules and hydrophilic adjuvants molecules.

2. The combined adjuvants nanoparticles according to claim 1, wherein, the combined adjuvants nanoparticles have diameters of 100-200 nm.

3. The combined adjuvants nanoparticles according to claim 1, wherein, the hydrophobic adjuvants molecules are monophosphatidyl lipid A or analogues thereof.

4. The combined adjuvants nanoparticles according to claim 1, wherein, the hydrophilic adjuvants molecules are oligonucleotides or oligodeoxynucleotides.

5. The combined adjuvants nanoparticles according to claim 4, wherein, the hydrophilic adjuvants molecules are CPG−ODN.

6. A preparation method for the combined adjuvants nanoparticles, wherein, it comprises the following steps:
S1: The monophosphatidyl lipid A or analogues thereof are modified with azide groups, and then subjected to dialysis and lyophilization;
S2: The resulting substances from S1 are mixed with oligodeoxynucleotides by stirring at room temperature for 12-18 h, and then subjected to dialysis and lyophilization to get the combined adjuvants nanoparticles.

7. The preparation method according to claim 6, wherein, in step S1, diphenylphosphoryl azide and 1,8-diazabicyclo[5.4.0]undec-7-ene are added for modification with azide groups, they are reacted with stirring at 20° C. for 24˜48 h and then subjected to dialysis and lyophilization; the ratio of the mass of monophosphatidyl lipid A, the volume of diphenylphosphoryl azide, and the volume of 1,8-diazabicyclo[5.4.0]undec-7-ene is (2.0˜4.0) mg:(3.0˜6.0) μl:(2.0˜4.0) μl; in step S2, the ratio of the mass of the resulting substances from S1 to the volume of oligodeoxynucleotides is (1.0˜2.0) mg: (100˜200) μl.

8. (canceled)

9. An immunogenic composition, wherein, comprising effective amounts of antigens and the combined adjuvants nanoparticles according to claim 1.

10. (cancelled)

11. An immunogenic composition, wherein, comprising effective amounts of antigens and the combined adjuvants nanoparticles according to claim 2.

12. An immunogenic composition, wherein, comprising effective amounts of antigens and the combined adjuvants nanoparticles according to claim 3.

13. An immunogenic composition, wherein, comprising effective amounts of antigens and the combined adjuvants nanoparticles according to claim 4.

14. An immunogenic composition, wherein, comprising effective amounts of antigens and the combined adjuvants nanoparticles according to claim 5.


SEQUENCE LISTING (1 sequence)

SEQ ID NO: 1 - DNA, 22 bp, Artificial Sequence
  oligodeoxyribonucleotides CPG-ODN
tcgtcgttttcggcgcgcgccg